(19) United States
(12) Patent Application Publication
(10) Pub. No.: US 2005/0085925 A1
(43) Pub. Date: Apr. 21, 2005

(54) Bio-Potential Activation of Artificial Muscles

(75) Inventors: Ethan A. Kottke, Farmington, NM (US); L. Donald Partridge, Albuquerque, NM (US)

Correspondence Address: MOHSEN SHAHINPOOR, 909 VIRGINIA, NE, SUITE 205, ALBERQUERQUE, NM, 87108, US

(73) Assignee: Mohsen Shahinpoor, Albuquerque, NM (US)

(21) Appl. No.: 10/605,676

(22) Filed: Oct. 16, 2003

(51) Int. Cl. A61F 2/70

(52) U.S. Cl. 623/25; 623/50

(57) ABSTRACT

The invention relates to the general field of electrical activation of non-biological artificial muscles, such as ionic polymeric synthetic artificial muscles, by means of action potentials produced by a biological nerve, such as mammalian sciatic nerve. This invention demonstrates how to stimulate and activate a non-biological muscle such as an ionic polymeric metal composite (IPMC) electro-active artificial muscle with the biological action potential generated by a mammalian nerve such as a rat sciatic nerve. The said invention further presents settings to generate optimal movement and force in artificial muscle due to the application of a nerve action potential. The invention uses the sciatic nerve to generate an action potential, which is subsequently amplified and applied to a cantilever sample of an electro-active ionic polymeric artificial muscle to cause it to bend, flex, and twitch. The sciatic nerve, in this invention, is stimulated by a separate signal to cause it to generate an action potential in the range of hundreds of μV, which is recorded by the electrodes attached to the nerve. These electrodes carry the action potential to an amplifier to amplify it to between 10's of Volts and subsequently are attached to the ionic polymeric artificial muscle to cause it to flex and twitch. Different frequencies of stimulation are tried to optimize the motion and force generated by the polymeric artificial muscles.

### BACKGROUND OF INVENTION

1. Field of the Invention

The invention relates to the general field of electrical activation of non-biological artificial muscles, such as ionic polymeric synthetic artificial muscles, by means of an action potential from a biological nerve, such as a mammalian sciatic nerve.

2. Related Prior Art

Muscles are complex and intricate parts of the human body. Without muscles, life in the current form would not be possible. Muscles assist in all human processes. Breathing, removal of wastes, eating, and locomotion, anything that a person does requires the use of muscles. Muscles can become damaged, be deficient, or suffer from dystrophy or atrophy. For example, ptosis is a muscle deficiency that leads to eyelid droop syndrome for many individuals , which cannot be entirely corrected surgically to the satisfaction of patients. There is definitely a need to be able to integrate artificial muscles with biological systems and to be able to utilize the motor neuron signals to actuate them. Artificial muscles can certainly use external power and the associated electronics as well, to function properly. However, making artificial muscles controllable by natural biological nerve action potentials will be an important improvement, as well as a challenge. This invention is about the methods and procedures to achieve functional integration and electrical activation of non-biological artificial muscles by means of biological nerve action potentials.

Artificial muscle discovery and use are rather new. Research into it has been conducted for less than 10 years. Research into the use of polymers began in the 1940s, but research into polymeric response to electrical stimulus only began in the mid 90s. Refer to the following classic review articles: M. Shahinpoor, Y. Bar-Cohen, J. Simpson and J. Smith, “Ionic Polymer-Metal Composites (IPMCs) As Biomimetic Sensors, Actuators and Artificial Muscles—A Review”, Smart Materials & Structures Int. Journal, vol. 7, pp. R15-R30, (1998), M. Shahinpoor, “Ionic Polymer-Conductor Composites As Biomimetic Sensors, Robotic Actuators and Artificial Muscles—A Review”, Electrochimica Acta, Vol. 48, No. 14-16, pp. 2343-2353, (2003), as well as M. Shahinpoor and M. Mojarrad, “Soft Actuators and Artificial Muscles,” U.S. Pat. No. 6,109,852, Issued Aug. 29, 2000, for more information on ionic polymeric synthetic artificial muscles. There are also other forms of artificial muscles, which are beyond the scope of the present invention. Amongst those one can mention shape-memory alloys (SMAs),shape-memory polymers (SMP's), magnetic shape-memory materials (MSM), electrochemically contractile ionic polymers such as polyacrylonitrile (PAN) as described by H. B. Schreyer, N. Gebhart, K. J. Kim, and M. Shahinpoor, “Electric Activation of Artificial Muscles Containing Polyacrylonitrile Gel Fibers”, Biomacromolecules, Vol. 1, No. 4, pp. 642-647, (2000), thermally contractile liquid crystal elastomer artificial muscle structures as described by Heino Finkelmann and M. Shahinpoor, “Electrically-Controllable Liquid Crystal Elastomer-Graphite Composites Artificial Muscles”, Proceeding of SPIE 9^{th} Annual International Symposium on Smart Structures and Materials, San Diego, Calif., SPIE Publication No. 4695-53, (March, 2002), magnetically deployable structures or other deployable structures equipped with smart materials such as piezocerams, piezopolymers, electro-active and electro-strictive polymers, magneto-strictive materials, and metal-hydride artificial muscles as described by M. Shahinpoor and K. J. Kim, “A Mega-Power Metal Hydride Anthropomorphic Biorobotic Actuator,”, Proceeding of SPIE 8 Annual International Symposium on Smart Structures and Materials, Newport Beach, Calif., Vol. 4327-(18) (March, 2001), in the patent entitled: “Metal Hydride Artificial Muscles”, U.S. Pat. No. 6,405,532, Issued Jun. 18, 2002 to M. Shahinpoor and K. J. Kim. The aforementioned artificial muscles are generally electrically activated by external electrical power and the associated electronics and robotic control. However, activating them with the bioelectric signals through nerve action potentials will be a new means of activation, and will have far reaching application consequences in biomedical engineering and medicine. In order to fully describe the present invention in connection with such nerve action potential activation of non-biological artificial muscles, it will be appropriate to briefly present a review on biological muscles, how they are activated, and the role that nerve action potentials play in this activation.

Biological muscle contraction begins when an electrical signal is generated from somewhere in the central nervous system, either as a voluntary activity from the brain or as a reflex activity from the spinal cord. A motor neuron located in the ventral horn of the spinal cord is then activated and an action potential passes through the neuronal axon to the innervated muscle through a ventral root of the spinal cord and peripheral nerve. The axon branches out to affect a number of muscle fibers, forming a motor unit. The motor neuron delivers the impulse to the motor end plates located on hundreds of muscle fibers. The brain recruits slow, low-force, fatigue resistant, motor units first, followed by fast, high-force, fatigable, motor units. This is because the slow twitch motor units will use less energy in a short-term muscle contraction than fast twitch motor units. Fast twitch motor units are used for “back-up”, for use after the slow-twitch motor units have been fatigued. (“Functional Basis of Muscle Contraction”, http://emile-21.com/VRML/muscleb1.html) and (“The Mechanism of Muscle Contraction”, http://savell-j.tamu.edu/muscontract.html). For a comprehensive reference on nervous systems, refer to Partridge, L. D., and Partridge, L. D., “Nervous System Actions and Interactions”, Kluwer Academic Press, Boston, Mass. (2003).

IPMC is a soft, lightweight, plastic-like material that is highly responsive electro-actively. It can mechanically bend under application of low voltages. This means that by applying a small voltage across this material (usually in the form of a membrane), one can induce a mechanical motion or deformation. Conversely, if IPMC is mechanically deformed or bent, a voltage can be generated from the material. In other words it acts as a sensor. The output voltage can easily be calibrated for any imposed force and displacement. This may enable IPMC to be used as biomedical sensors to detect loads or displacements of joints in the limbs or other organs in the human body.

Certainly an important potential area for the application of synthetic muscle is its biomedical uses. One such application in the near future might be small, surgically implanted pills that can periodically disperse medication for a patient. The implanted pills or “capsules” are perforated with microscopic holes to dispense the medication. A tiny ring of artificial muscle guards each hole. Medication will be dispensed by contractions in the synthetic muscle caused by electrical stimulus. Use of this type of pill could be with diabetics and their need for timed release of insulin. The pill, when empty, can then be surgically removed and a new one implanted. If such medication release is performed under neuronal control by an action potential, it will be a breakthrough in medication management.

A more detailed explanation will follow in the specific description of the invention later. It should be mentioned that the use of action potentials to activate an IPMC is very promising. While the IPMC needs an electrical stimulus to begin the ionic displacement necessary for it to move, the signal of an action potential has the appropriate properties to excite the IPMC. However, action potentials by themselves are not of sufficient voltage to cause IPMC to displace. An action potential can easily be amplified and applied to IPMC, causing predictable and repeatable displacement. Utilizing nerves to power the IPMC adds the important possibility of biological control of IPMCs for use as an artificial muscle. This new, cutting edge, versatile technology has the potential to assist cardiac, intestinal, paralysis, and ocular patients, among others. Stronger, more efficient and more robust electro-active polymers are currently being made to combat practical problems such as consuming too much energy, not lasting long enough, and insufficient force being generated.

For example, for ptosis, or the eyelid droop syndrome, a flexing sheet of IPMC muscle would be integrated with the eyelid and electronically, remotely activated to blink the eyelid approximately every second or so. Battery power has been considered, but because it is not generally biocompatible, it is not very promising. More compact and efficient methods of powering the synthetic muscle are being sought out. One of these methods is utilizing nerve action potentials from neighboring areas of the implantation.

In current breakthrough research, electro-active polymers have been created that require only one-tenth the voltage previously needed. They are highly efficient and have a fast response. IPMC technology integrated with nerve action potentials is the main subject of the present invention.

A primary object of the present invention is to create on-demand activation of artificial muscles by means of biological nerve action potentials.

Another object of the present invention is to create an active “smart system” that responds to biological nerve action potentials in a dynamic manner.

Yet another object of the present invention, is to provide a biomedical engineering system for activation of artificial and/or synthetic muscles, robots, and devices by means of biological nerve action potentials.

An advantage of the present invention is that it facilitates a surgical methodology and technology that enables the integration and implantation of artificial muscle in biological muscular systems. This can correct muscular atrophy, dystrophy, disability, and the muscle weakness and deficiencies seen in the elderly and disabled individuals who have lost control over their muscles.

Other objects, advantages, novel features, and further scope of applicability of the present invention will be set forth in part in the detailed description to follow. This description, taken in conjunction with the accompanying drawings, will in part become apparent to those familiar with the art upon examination of the following, or by practice of the invention. The objects and advantages of the invention may be realized and attained by means of the instrumentalities and combinations particularly pointed out in the claims.

### SUMMARY OF INVENTION

This invention concerns a method to stimulate and activate a non-biological muscle such as an ionic polymeric metal composite (IPMC) electro-active artificial muscle with the biological action potential generated by a mammalian nerve such as the sciatic nerve. The invention further presents settings to generate optimal movement and force in artificial muscle due to the application of a nerve action potential. The invention uses the sciatic nerve of a rat to generate an action potential, which is subsequently amplified and applied to a cantilever sample of an electroactive ionic polymeric artificial muscle to cause it to bend, flex and twitch. The rat sciatic nerve was rapidly removed from a euthanized animal by procedures that were in accordance with the guidelines of the University of New Mexico Medical School and the National Institutes of Health. The nerve was placed in a nerve bath where it made contact with silver recording and stimulating electrodes. Synchronous action potentials were generated with brief electrical pulses from a pulse generator and the resultant action potential was propagated to the recording electrodes, where it was recorded after appropriate amplification. The extra-cellularly recorded compound action potential was a few hundred μV and it was amplified to between 20-40 Volts. It was subsequently input to electrodes integrated with a sample of ionic polymeric artificial muscles (IPMC's) to cause it to flex and twitch. Different frequencies of stimulation were tried to optimize the motion and force generated by the polymeric artificial muscles.

### BRIEF DESCRIPTION OF DRAWINGS

The accompanying drawings, which are incorporated into and form a part of the specification, illustrate several embodiments of the present invention and, together with the description, serve to explain the principles of the invention. The drawings are only for the purpose of illustrating a preferred embodiment of the invention and are not to be construed as limiting the invention. In the drawings:

FIG. 1 is a view of the rat sciatic nerve in the nerve bath with electrodes attached to it.

FIG. 2 is a view of a typical compound action potential from the sciatic nerve.

FIG. 3 is a view of typical dynamic force generated by the polymeric artificial muscle stimulated by an amplified action potential from the stimulated sciatic nerve.

### DETAILED DESCRIPTION

As discussed before, the present invention demonstrates how to stimulate and activate a non-biological muscle such as an ionic polymeric metal composite (IPMC) electro-active artificial muscle with the biological action potential generated by a mammalian nerve such as a sciatic nerve. The invention further presents parameters to generate optimal movement and force in artificial muscle due to the application of a nerve action potential. In order to fully describe the present invention in connection with such nerve action potential activation of non-biological artificial muscles, one should recall the review presented in the previous background section on biological muscles, how they are activated, and what role the nerve action potential plays in their activation. As discussed before, biological muscle contraction begins when an electrical signal is generated from somewhere in the central nervous system, either as a voluntary activity from the brain or as a reflex activity from the spinal cord and an action potential is propagated through a peripheral nerve to muscle fibers of a motor unit.

There is no actual (cytoplasmic) connection between motor neurons and skeletal muscle fibers. When a motor neuron depolarizes, an electrical current (the action potential) propagates as a wave along the fiber. When the impulse reaches the motor end plate (the end of the neuron), the action potential causes the release of packets (quanta) of acetylcholine (the neurotransmitter) into the synaptic clefts between the nerve and the muscle fiber and the acetylcholine binds to receptors on the muscle membrane. Acetylcholine causes the electrical resting potential under the motor end plate to change, initiating an action potential that passes in both directions along the surface of the muscle fiber. An enzyme, acetylcholinesterase, breaks down the released acetylcholine to prevent a continued action of acetylcholine at the muscle membrane receptors. This insures that the nerve is in complete electrical control of the muscle. The muscle action potential reaches the transverse tubules through which the action potential spreads into the muscle fiber. At each point where a transverse tubule touches part of the sarcoplasmic reticulum, it causes the sarcoplasmic reticulum to release Ca²⁺ ions. This results in movement of troponin and tropomyosin on the thin filaments of the muscle fiber. Troponin and tropomyosin are two proteins that form a blocker between actin and myosin molecules that keep the two other proteins from interacting with each other. The presence of the Ca²⁺ allows for the interaction between actin and myosin. When Ca²⁺ is present, the shape of the troponin-tropomyosin complex changes, and now actin and myosin can come into contact with each other. This enables the myosin molecule heads to “grab and swivel” their way along the actin filament, which is the driving force of muscle contraction. This process is known as the “sliding filament theory of muscle contraction”, and was initially proposed by Noble laureate A. F. Huxley in 1957. (Freudenrich, Craig C., PhD “How Muscles Work: Triggering Contraction”, http://www.howstuffworks.com/muscle4.htm, ©1998-2001 Howstuffworks, Inc.), (“NISMAT Exercise Physiology Corner: A Primer on Muscle Physiology”, http://www.nismat.org/physcor/muscle.html, ©1996-2000 The Nicholas Institute of Sports Medicine and Athletic Trauma, Partridge, L. D., and Partridge, L. D., “Nervous System Actions and Interactions”, Kluwer Academic Press, Boston, Mass., (2003).

Energy for the reorientation of the myosin molecule heads comes from ATP. Oddly enough though, it also takes energy to stop the process. Muscle contraction stops when Ca²⁺ is removed from the immediate vicinity of the myofilaments. The sarcoplasmic reticulum actively pumps Ca²⁺ back into itself and this requires utilization of ATP. Troponin-tropomyosin re-assume their inhibitory position between the actin and myosin molecules once Ca²⁺ is removed. The muscle is now relaxed and no more energy is being used.

Just as electrical signals are a big part of biological muscle contraction, electricity is the motivating force behind electro-active artificial muscle contraction. To start the process of contraction, an electrical stimulus is applied to the artificial muscle. This stimulus, X, is in the range 0<X<8 volts. Frequency can also affect the movement of the artificial muscle by affecting the muscle's rate of displacement. The higher the frequency, the faster the artificial muscle will move, and vice versa. Electricity causes the mobile charges within the artificial muscle that have opposite polarities to sort themselves on opposite sides of the actuator. The actuator of the muscle is the polymer contained between the two sheets of metal (Platinum, Palladium or Gold work well as the metal, as they are very conductive to electrical stimulus and do not corrode in biological fluids). (Czerniecki, Joseph M., Hannaford, Blake, and Klute, Glenn K. “McKibbenn Artificial Muscles: Pneumatic Actuators with Biomechanical Intelligence”, http://rcs.ee.washington.edu/BRL/, IEEE/ASME 1999 International Conference on Advanced Intelligence Mechatronics, (AIM '99) Sept. 19-22, 1999 in Atlanta, Ga.) and (DeGennes, P. G., Kim, K. J., Okumura, Ko, and Shahinpoor, M., Published in Europhysics Letters vol. 50, no. 4 pp. 513-518, 2000, http://www.me.unm.edu/˜shah/).

Synthetic muscle contraction is quite similar to the way a biological muscle contracts. The electrical stimulus applied to the synthetic muscle takes the place of not only the nerves of the biological muscle, but also the Ca²⁺. These initiate biological muscle contraction by allowing the interaction between troponin and tropomyosin. The mobile charges of the synthetic muscle are analogous to the actin and myosin molecules in that they attract to encourage contraction of the muscle, but separate in order to relax the muscle. Finally, the Carbon and Fluorine molecules are similar to the troponin and tropomyosin in that they both keep the chemicals that cause contraction, separated.

The nervous system contains vast circuits of delicate cells called neurons that are elaborately interconnected. Most neurons conduct impulses along hair-like cytoplasmic extensions called axons. Each neuron has just one axon that can range from a few millimeters to an entire meter in length, but are only a few micrometers in diameter. Axons grow out of the cell body, which houses the organelles needed by the nerve to stay alive and there is a steady transport of materials from the cell body along the entire length of the axon. Short, branched fibers called dendrites extend from the cell body. Nerve impulses are usually generated from inputs to dendrites and conducted through the rest of the neuron. Many axons are covered with a glistening fatty sheath called the myelin sheath. The sheath is the greatly expanded plasma membrane of an accessory cell called the Schwann cell. Schwann cells are spaced evenly along the axon, with their plasma membranes wrapped around the axon forming the myelin sheath. The gaps between the Schwann cells are unprotected and these regions, called the node of Ranvier, play an important role in the propagation of the nerve impulse. Neurons are specialized with specific functions to take in sensory information, control muscles, and to allow our brains to learn, reason, and remember. Each neuron has tens of thousands of synapses that it establishes and maintains. Synapses are the microscopic spaces between the axon of one nerve cell and the dendrite of another. The synapse encompasses the end of the axon, the beginning of the dendrite, and the synaptic cleft (the space between the axon and the dendrite). (“Lights, Camera, Action Potential!”,http://faculty.washington.edu/chudler/ap.html) and (“Neurons”,http://users.rcn.com/jkimball.ma.ultranet/BiologyPages/N/Neurons.html,©June, 2001).

When a neuron is not sending a signal, it is said to be “at rest”. When a neuron is “at rest”, it is negative on the inside with respect to the outside. The membrane of the neuron is semi-permeable because of specific ion channels that control the crossing of ions through the membrane. Regulation of the crossing of the ions is necessary to control the potential of the inside of the neuron. At rest, potassium ions (K⁺) can cross through the membrane easily, chloride (Cl⁻) and sodium (Na⁺) ions have a difficult time crossing, and the negatively charged protein molecules (A⁻) inside the neuron cannot cross the membrane. The sodium-potassium pump transports three Na⁺ ions out of the neuron for every two K⁺ ions transported into the cell. When all of these charges balance out, the resting membrane potential can be measured. The resting membrane potential is around −70 mV, meaning that the inside of the neuron is 70 mV more negative than the outside. This is due to the selective outward movement of K⁺ leaving behind an excess of immobile anions (A⁻). While the resting potential exists when the neuron is at rest, the action potential occurs when the neuron relays a signal down the axon, away from the cell body. An action potential is a rapid transient change in potential that is used to signal information in the nervous system (and in muscles). The first stage of an action potential is depolarization resulting from the increased movement of Na⁺ ions across the neuron membrane into the neuron. Ion channels open and allow for Na⁺ to diffuse into the cell at high rates. Sodium ions carry a positive charge with them and cause the membrane potential of the neuron to depolarize. As more sodium channels open, the membrane potential of the neuron reaches around +30 mV, but will then proceed to return to its resting potential. Sodium channels now begin to close and potassium channels begin to open. Due to the excess of K⁺ in the neuron, K⁺ ions rush out from the neuron and cause it to become increasingly negative. The potassium channels remain open for a long enough that the neuron experiences a period of increased hyper-polarization. The potassium channels subsequently close and the neuron gradually returns to the resting level and the potential becomes −70 mV once more. Action potentials are initiated when the membrane potential reaches a threshold level of about −55 mV. If the membrane potential does not reach this level, no action potential will fire. This is the “All-or-None” principle. No matter how much the neuron is stimulated over threshold, the same size action potential will always be generated. A neuron is usually stimulated at its dendrites or cell body. For the action potential to be propagated down the nerve, it is regenerated sequentially as a wave along the axon. It is quite similar to the domino effect in that once the first action potential has been generated; its strong depolarization causes an action potential to occur in a neighboring region of the axon. This continues until it reaches the end of the axon. Enough axons end on a single neuron cell body or dendrite to cover most of the cell body. The number of axons ending on a single neuron provides the morphological basis for the combination of actions of different nerve fibers. Action potentials must often be transferred from one neuron to another. The synapse is a unique junction that controls communication between neurons. There are two types of synapses: electrical and chemical. An electrical synapse allows action potentials to spread directly from presynaptic cells to postsynaptic cells through structures called gap junctions, which are intercellular channels that allow the ions to flow directly from cell to cell. The other form of synapse is a chemical synapse. At a chemical synapse, a narrow gap, or a synaptic cleft, separates the presynaptic cell from the postsynaptic cell. Due to the cleft, a signal cannot be immediately transferred between two cells. The key to understanding the function of a chemical synapse is its structure. Within the cytoplasm of the synaptic terminal are numerous sacs called synaptic vesicles. Each of these sacs contains thousands of molecules of a neurotransmitter, which is the messenger that crosses the synaptic cleft. Most neurons secrete only one kind of transmitter, but most neurons can accept many different types of neurotransmitter. When an action potential arrives at the synaptic terminal, the presynaptic cell releases neurotransmitters into the synapse. The presynaptic membrane is then depolarized, causing the synaptic vesicles to fuse with it, releasing the neurotransmitters into the synaptic cleft through exocytosis. The neurotransmitter molecules diffuse across the synaptic cleft and bind to receptors on the postsynaptic membrane. These receptors are associated with ion channels that allow certain ions to cross the postsynaptic membrane. The neurotransmitters are quickly disintegrated by enzymes, and the action potential continues along the postsynaptic membrane. (Neil A. Campbell, “Biology: Fourth Edition”, Chapter 44, Benjamin/Cummings Publishing Company, Inc., Menlo Park, Calif., (1996)) and (“Lights, Camera, Action Potential!”,http://faculty.washington.edu/chudler/ap.html, Partridge, L. D., and Partridge, L. D., “Nervous System Actions and Interactions”, Kluwer Academic Press, Boston, Mass., (2003).

A possible problem with the stimulation of IPMC by action potentials is that action potentials firing in the axons that make up a peripheral nerve are asynchronous. The experiments leading to this invention were performed by causing synchronous firing of action potentials in all of the fibers in a nerve and this was an effective means of activating the IPMC. Future experiments will need to be performed to test the feasibility of using asynchronous firings in activating the IPMC.

The integration of action potentials with IPMC is very promising. While IPMC needs an electrical stimulus to begin the ionic displacement necessary for it to move, the signal of an action potential has the appropriate properties to excite the IPMC. However, action potentials by themselves do not produce enough voltage to cause an IPMC to displace much. An action potential can easily be amplified and applied to IPMC, causing predictable and repeatable displacement. Utilizing nerves to power the IPMC adds the important possibility of biological control of IPMCs in use as an artificial muscle. This new, cutting edge, versatile technology has the potential to assist cardiac, intestinal, paralysis, and ocular patients, among others. Stronger, more efficient and more robust electro-active polymers are currently being made to combat practical problems such as consuming too much energy, not lasting long enough and insufficient force being generated. In current breakthrough research, electro-active polymers have been created that require only one-tenth the voltage previously needed. They are highly efficient and have a fast response. IPMC integrated with nerve action potentials is the main subject of the present invention.

The said invention, thus, uses the sciatic nerve of a rat to generate an action potential, which is subsequently amplified and applied to a cantilever sample of an electro-active ionic polymeric artificial muscle to cause it to bend, flex, and twitch. The rat sciatic nerve was rapidly removed from a euthanized animal by procedures that were in accordance with guidelines of the University of New Mexico and the National Institutes of Health. The nerve was placed in a nerve bath where it made contact with silver recording and stimulating electrodes. Synchronous action potentials were generated with brief electrical pulses from a pulse generator and the resultant action potential was propagated to the recording electrodes where it was recorded after appropriate amplification. The extracellularly recorded compound action potential was a few hundred μV and it was amplified to between 20-40 Volts and subsequently attached to the ionic polymeric artificial muscle to cause it to flex and twitch. Different frequencies of stimulation were tried to optimize the motion and the force generated by the polymeric artificial muscles.

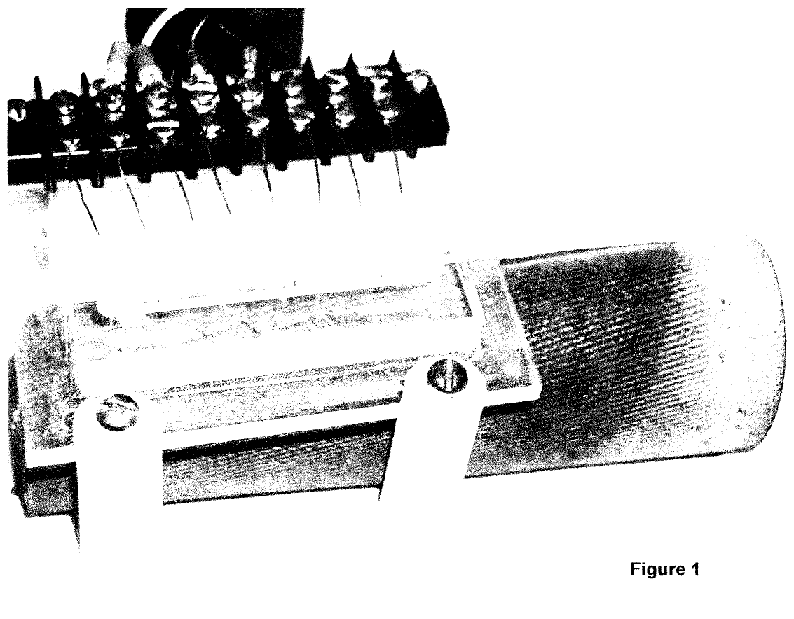

FIG. 1 is a view of the rat sciatic nerve on the nerve bath with electrodes attached to it. The nerve tissue was placed on a nerve bath, and using a signal generator, various signals were generated and compound action potentials recorded to find the optimum stimulation settings for the mammalian nerve. The anode was placed 1 cm from the cathode, so that the generated action potential was not mixed with the excitation signal. In between trials, the nerve was re-hydrated in Ringer's solution for one minute to ensure proper action potential generation.

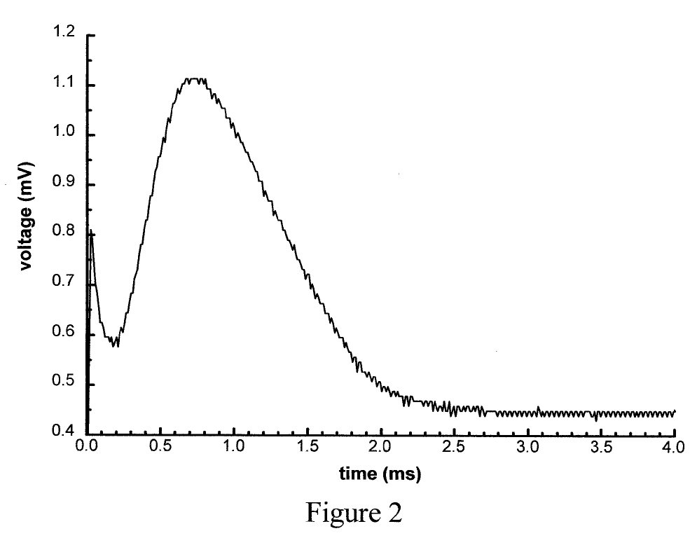

FIG. 2 is a view of a typical compound action potential from the sciatic nerve. Several different action potentials were recorded to ensure accuracy. The action potentials were then averaged and the average was graphed, as shown.

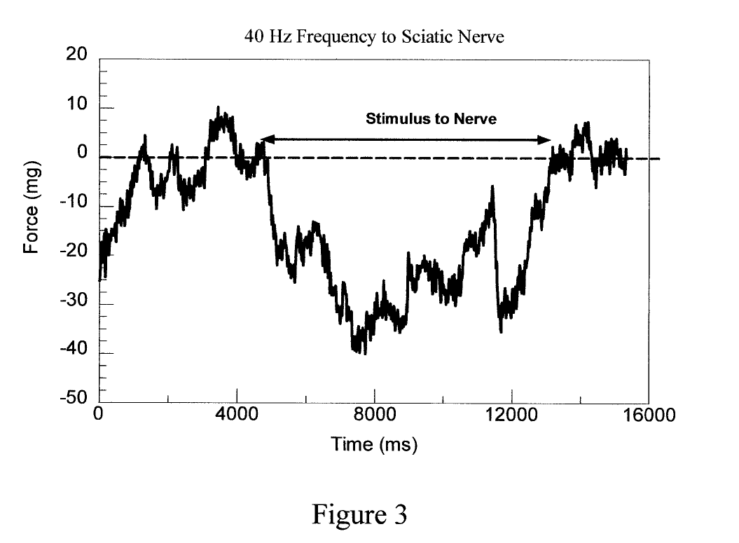

FIG. 3 is a view of typical dynamic force generated by the polymeric artificial muscle activated by the amplified action potentials from the sciatic nerve. This graph demonstrates the force with which the IPMC displaces when action potentials at 40 Hz were applied to it. As shown on the graph, the force is relatively constant until the 5 second (5000 ms) mark (action potentials were applied to the IPMC at this time), when it drops off suddenly. The force is then increased for 5 more seconds (5000 ms), and it abruptly decreases back to its original state when the nerve stimulation is terminated and action potentials are no longer delivered to the IPMC.

1- Procedures and methods for electrical actuation of non-biological artificial muscles, such as ionic polymeric synthetic artificial muscles and electroactive polymers, by means of action potential generated by biological nerve, such as mammalian sciatic nerve:

2- Procedures and methods for using the nerve of a biological system or entity to generate an action potential, which is subsequently, amplified and applied to a sample of an electro-active ionic polymeric artificial muscle to cause it to bend, flex, deform, stretch, contract and twitch.

3- Procedures and methods for using the nerve of a biological system or entity to generate an action potential, which is subsequently, amplified and applied to a sample of an electro-active artificial muscle, material or system to cause them to deform, change shape, become dynamically active, bend, flex, stretch, contract and twitch.
